(19) United States
(12) Patent Application Publication
(10) Pub. No.: US 2022/0040378 A1
(43) Pub. Date: Feb. 10, 2022

(54) WOUND CARE TREATMENT AND METHODS OF MAKING AND USING SAME

(71) Applicant: Prime Merger Sub, LLC, Birmingham, AL (US)

(72) Inventors: Steven J. THOMAS, Birmingham, AL (US); Katie C. Mowry, Birmingham, AL (US)

(21) Appl. No.: 17/508,958

(22) Filed: Oct. 22, 2021

Related U.S. Application Data
(63) Continuation of application No. 15/309,566, filed on Nov. 8, 2016, now abandoned.
(60) Provisional application No. 61/993,089, filed on May 14, 2014.
(60) Provisional application No. 62/006,986, filed on Jun. 3, 2014.

(51) Int. Cl. A61L 27/38; A61L 15/32; A61L 15/40; A61L 27/24; A61L 27/26; A61L 27/36; A61L 27/56

(52) U.S. Cl. CPC A61L 27/3804; A61L 15/32; A61L 15/40; A61L 27/24; A61L 27/26; A61L 2300/64; A61L 27/3691; A61L 27/3839; A61L 27/56; A61L 2300/414; A61L 27/3633

(57) ABSTRACT

A method of treating a wound including applying a wound care treatment to the wound, the wound care treatment including a preparation composed of morselized amnion tissue and amniotic fluid cells adsorbed to a porous collagen matrix and optionally, glycosaminoglycan. The morselized amnion tissue includes organized amniotic extracellular matrix (ECM), amniotic tissue cells and growth factors contained within the ECM and amniotic tissue cells. The porous collagen matrix is provided as a solid sheet, a meshed or perforated sheet or a flowable material.

### RELATED APPLICATIONS

This application claims priority to U.S. Provisional Patent Application 61/993,089, filed on May 14, 2014; U.S. Provisional Patent Application 62/006,986, filed on Jun. 3, 2014, PCT/US15/30717 filed on May 14, 2015, and U.S. Non-provisional patent application Ser. No. 15/309,566 filed on Nov. 8, 2016, all titled, “Wound Care Treatment and Methods of Making and Using Same,” the entire contents of which are incorporated herein by reference.

### TECHNICAL FIELD

The present invention is directed to a wound care treatment. More particularly, the present invention is directed to a wound care treatment for promoting wound healing, the treatment including adsorbing amniotic tissue cells and particles into a porous collagen matrix and applying the preparation to a wound or damaged or diseased tissue.

### BACKGROUND OF THE INVENTION

Wound healing is a complex process where the skin or another organ or tissue repairs itself after injury. In normal skin, the epidermis (outermost layer) and dermis (inner or deeper layer) form a protective barrier against the external environment. If the protective barrier is broken, the normal process of wound healing is immediately set in motion. Upon injury to the skin, a set of complex biochemical events takes place to repair the damage. The classic model of wound healing is divided into several sequential, yet overlapping phases. Effective wound healing requires the highly organized integration of complex molecular and biological events including cell proliferation, migration and extracellular matrix (ECM) deposition. The speed of wound healing can be impacted by many factors, including the health of the individual and the bloodstream levels of certain hormones.

The wound healing process is not only complex but fragile, and susceptible to interruption or failure leading to the formation of non-healing chronic wounds. A non-healing wound is one that fails to heal with standard therapy in an orderly and timely manner (Troxler, M. et al., “Integrating adjunctive therapy into practice: the importance of recognizing ‘hard-to-heal’ wounds.” World Wide Wounds 2006. Available from http://www.worldwidewounds.com/2006/december/Troxler/Integrating-Adjunct-Therapy-Into-Practice.html). One of the major factors responsible for the appearance of chronic wounds is the impairment of cytokine release by local fibroblasts and inflammatory cells, which can result in reduced angiogenesis (Falange, V. (2005) “Wound healing and its impairment in the diabetic foot,” Lancet 366: 2736-1743). Many health-related factors may contribute to the development of non-healing wounds, including immunological diseases, diabetes, venous or arterial disease, advanced age, and infection.

Healing may be promoted by restoring or preventing the breakdown of the skin or tissue/organ extracellular matrix. This may be accomplished through the addition of deficient components, such as growth factors or collagen, or the introduction of a temporary matrix to support the growth of new cells or tissue. Regenerative therapies involve the use of living cells to repair, replace or restore normal function to damaged tissues and organs. Stem cells are viewed as a promising candidate for use in cell-based wound healing therapies due to their capacity for self-renewal and differentiation. Both adult and embryonic stem cells are commonly used to develop therapies for various models of disease and injury. However, a number of limitations hamper the clinical applicability of stem cells derived from adults or developing embryos, such as ethical concerns and limitations on the cell sample size.

Subpopulations of stem cells exist in both the amniotic membrane and the amniotic fluid. Amniotic fluid cells are obtained during amniocentesis or scheduled C-section while amniotic membrane cells are obtained from the amnion membrane which is discarded after birth. These cells are therefore readily available, easily procured and avoid the ethical issues surrounding the use of embryonic stem cells.

Human amniotic fluid is a dynamic environment, which undergoes multiple developmental changes in order to sustain fetal growth. Fluid secretions from the fetus into the amniotic fluid carry a variety of fetal cells, resulting in a heterogeneous population of cells derived from fetal skin, gastrointestinal, respiratory and urinary tracts, and the amniotic membrane. These cells express electrolytes, growth factors, carbohydrates, lipids, proteins, amino acids, lactate, pyruvate, enzymes, hormones and other factors useful in tissue repair. Because they are readily accessible and pose little to no ethical concerns, amniotic fluid-derived cells are a promising alternative source of cells for use a strategy for cell replacement in various injury models. U.S. Patent Application Publication No. 2010/0130415 (Cohen et al.) describes formulations comprising secreted products obtained from the culture medium of stem cells, such as umbilical cord blood stem cells, for enhancement of wound healing.

Clinicians have used intact placental membrane, comprising an amnion and a chorion layer, in medical procedures since as early as 1910 (Davis, J. S., John Hopkins Med. J. 15, 307 (1910)). The amniotic membrane, when separated from the intact placental membrane, may also be used for its beneficial clinical properties (Niknejad H, et al. Eur Cell Mater 15, 88-99 (2008)). Certain characteristics of the placental membrane make it attractive for use by the medical community. These characteristics include, but are not limited to, its anti-adhesive, anti-microbial, and anti-inflammatory properties; wound protection; ability to induce epithelialization; and pain reduction. (Mermet I, et al. Wound Repair and Regeneration, 15:459 (2007)).

The placental membrane has a wide number of applications in regenerative medicine, including providing scaffolding or structure for the regrowth of cells and tissue. An important advantage of placental membrane in scaffolding is that the amnion contains an epithelial layer. The epithelial cells derived from this layer are similar to stem cells, allowing the cells to differentiate into cells of the type that surrounds them. Additional cells similar to stem cells are contained in the body of the membrane, and the membrane also contains various growth and trophic factors, such as epidermal, insulin-like and fibroblast growth factors, and high concentrations of hyaluronic acid which may be beneficial in preventing scarring and inflammation and supporting healing. Thus, placental membrane offers a wide-variety of advantages for medical uses.

Although placental membranes possess many benefits and applications, availability of the membranes has limited their use. The amount of placental membrane generated from a single birth is small. As would be expected, because the supply of placental membranes is relatively small, the cost of placental membranes limits their use only to procedures that surpass a certain price or complexity. Additionally, membrane availability and cost limits the feasibility of amniotic membrane in the treatment of larger wounds. Accordingly, there is a need for means of increasing the effective supply of placental membranes and for preparations which will allow the delivery of the benefits of the placental membranes across a variety of applications. This is especially true in wound-healing applications where wounds may exhibit large surface areas, for example, wounds caused by chemical or heat-related burns.

### SUMMARY OF THE INVENTION

The present invention is directed to a preparation and treatment for promoting healing of wounds and damaged tissue including burns, abrasions, lacerations, abscesses, diseased tissue and the like. In one embodiment, the treatment includes providing an amniotic tissue preparation including a liquid such as saline, amnion tissue and amniotic fluid cells, wherein the amnion tissue is ground amnion tissue and includes organized amniotic extracellular matrix (ECM), amniotic tissue cells and growth factors contained within the ECM and amniotic tissue cells. The ECM can include amnion-derived collagen derived from an epithelium layer, a basement membrane layer, a compact layer, a fibroblast layer, an intermediate layer and a spongy layer of the amnion tissue. The ECM can also include fibronectin, laminin, hyaluronic acid, proteoglycans and glycosaminoglycans. A suitable amniotic tissue preparation is sold by NuTech Medical, Inc. of Birmingham, Ala. under the name NuCel™.

The amniotic tissue preparation is combined with a porous matrix including a mammalian collagen and optionally, glycosaminoglycans (GAG) or proteoglycans, to form a wound care preparation including a portion of the amniotic tissue preparation adsorbed to the porous matrix. The collagen matrix serves as a carrier for the amniotic fluid cells and morselized placental membrane of the amniotic tissue and acts to uniformly disperse the amniotic fluid cells and morselized placental membrane over a wound. Because the placental membrane is morselized, it can effectively treat a larger wound surface area than if the placental membrane were applied directly to a wound in solid form. For example, depending on the concentration of morselized placental membrane and amniotic fluid cells within the amniotic tissue and the amount of adsorption thereof into the collagen matrix, the wound surface area treated using the wound care preparation of the present invention can be between at least twenty times larger or between ten to two hundred times larger than if the placental membrane were applied directly to a wound in solid form.

Depending on the wound, the mammalian collagen of the collagen matrix can be provided as a sheet or a meshed sheet, when the wound care preparation is to be applied to a wound surface, or a flowable matrix, when the wound care preparation is to be injected into a wound bed or applied to a wound surface. The collagen may contain Type I collagen, Type III collagen, a granulated cross-linked tendon collagen or combinations thereof. It is important that the collagen matrix retains an amount of amniotic tissue that is sufficient to effectively treat a wound based on the size of such wound. For example, when the porous matrix is provided as a sheet, the amniotic tissue preparation adsorbs to the sheet in an effective amount ranging from about 300 μl to about 999 μl per square inch of the sheet. Suitable collagen matrices are sold by Integra Life Sciences Corporation of Plainsboro, N.J. under the names TenoGlide®, which is provided as a sheet, and Integra™, which is provided as a dermal template sheet and a flowable matrix. Suitable collagen matrix sheets, both meshed and solid are available from TEI Biosciences of Waltham, Mass. under the name PriMatrix™.

The wound care preparation is applied to a wound of a mammal in an effective amount for promoting wound healing. The wound care preparation can be applied topically, or it may be injected into a wound or wound bed when the flowable collagen matrix is used. An effective amount of the second preparation delivers between about 0.05 mg to about 5 mg, between about 0.1 mg to about 2.5 mg or between about 0.1 mg to about 1 mg of amnion tissue per square centimeter of the wound. The wound care preparation can be applied to a wound soon after it is prepared. Thus, for example, the wound care preparation can be applied immediately after the wound care preparation is prepared, or it can be applied to the wound within about 10 minutes of being prepared, about 30 minutes of being formed or several hours after being prepared. Further, the amniotic tissue preparation can be shipped and stored separately from the collagen matrix.

In a second embodiment, the treatment includes a porous collagen matrix and an amniotic tissue preparation adsorbed to the porous collagen matrix. In particular, the amniotic tissue preparation includes amnion tissue and amniotic fluid cells, wherein the amnion tissue is ground amnion tissue and includes amniotic extracellular matrix (ECM), amniotic tissue cells and growth factors and wherein the ECM includes fibronectin, laminin, proteoglycans, glycosaminoglycans and amnion-derived collagen that is derived from an epithelium layer, a basement membrane layer, a compact layer, a fibroblast layer, an intermediate and a spongy layer of the amnion tissue. The porous collagen matrix can be provided in the form of a sheet, meshed sheet or a flowable, powdered material.

### BRIEF DESCRIPTION OF THE DRAWINGS

FIG. 1 is a graph comparing adsorption amounts of a saline suspension containing NuCel™ amniotic tissue and a phosphate buffered saline (PBS) solution to a 1×1 inch square of PriMatrix™ collagen matrix.

FIG. 2 is a graph illustrating the respective percentages of saline and the particulate component (amniotic fluid cells and morselized placental membrane) of the saline solution containing NuCel™ amniotic tissue that adsorbs to a 1×1 inch square of PriMatrix™ collagen matrix.

FIG. 3 is a table illustrating adsorption amounts of a saline solution containing NuCel™ amniotic tissue to three 1×1 inch square samples of TenoGlide® collagen matrix.

FIG. 4 is a table illustrating the respective percentages of saline and the particulate component (amniotic fluid cells and morselized placental membrane) of the saline solution containing NuCel™ amniotic tissue that adsorbs to a 1×1 inch square of TenoGlide® collagen matrix.

FIG. 5 depicts 10× and 20× bright field, fluorescent and merged images showing 4′,6-diamidino-2-phenylindole (DAPI) labeled amniotic fluid cells and morselized placental membrane particles of the saline solution containing NuCel™ amniotic tissue adsorbed to the TenoGlide® collagen matrix.

FIGS. 6A and 6B are photographs depicting use of a double syringe mechanism for mixing Integra™ flowable collagen matrix with a saline solution containing NuCel™ amniotic tissue.

FIG. 7 depicts 10× and 20× bright field, fluorescent and merged images showing DAPI labeled amniotic fluid cells and morselized placental membrane particles of the saline solution containing NuCel amniotic tissue adsorbed to the Integra™ flowable collagen matrix.

### DETAILED DESCRIPTION

Before the present compositions, articles, devices, and/or methods are disclosed and described, it is to be understood that they are not limited to specific methods unless otherwise specified, or to particular reagents unless otherwise specified, and as such may vary. It is also to be understood that the terminology as used herein is used only for the purpose of describing particular embodiments and is not intended to be limiting.

This application references various publications. The disclosures of these publications, in their entireties, are hereby incorporated by reference into this application to describe more fully the state of the art to which this application pertains. The references disclosed are also individually and specifically incorporated herein by reference for material contained within them that is discussed in the sentence in which the reference is relied upon.

### A. Definitions

In this specification, and in the claims that follow, reference is made to a number of terms that shall be defined to have the following meanings:

As used herein, ranges can be expressed as from “about” one particular value, and/or to “about” another particular value. When such a range is expressed, an embodiment includes from the one particular value and/or to the other particular value. Similarly, when values are expressed as approximations, by the use of “about,” it will be understood that the particular value forms another embodiment. It will be understood that the endpoints of each of the ranges are significant both in relation to the other endpoint and independently of the other endpoint. It will also be also understood that there are a number of values disclosed herein, and that each value is also disclosed herein as “about” that particular value in addition to the value itself. For example, if the value “50” is disclosed, then “about 50” is also disclosed. It is also understood that when a value is disclosed that “less than or equal to” a value, that values “greater than or equal to the value” and possible ranges between values are also disclosed, as understood by one skilled in the art. For example, if the value “50” is disclosed, then “less than or equal to 50” and “greater than or equal to 50” are also disclosed. It is also understood that the throughout the application, data are provided in different formats, and it is understood that these data represent endpoints and starting points as well as ranges for any combination of the data points. For example, if a particular data point “50” and a particular data point “100” are disclosed, it is understood that greater than, greater than or equal to, less than, less than or equal to, and equal to 50 and 100 are considered disclosed as well as between 50 and 100.

As used herein, “amniotic fluid cells” mean cells that have been extracted, retrieved or derived from amniotic fluid from an amniotic sac of a pregnant female.

As used herein, “amniotic tissue” means amniotic fluid cells, placental membrane, amnion tissue or combinations thereof.

As used herein, the terms “optional” or “optionally” mean that the subsequently described event or circumstance may or may not occur, and that the description includes instances where said event or circumstance occurs and instances where it does not occur.

As used herein, the phrases “placental membrane” or “amnion tissue” refer to one or more layers of the placental membrane. For example, placental membrane or amnion tissue may refer to a placental membrane comprising both the amniotic and chorionic layers. In another example, placental membrane or amnion tissue may refer to a placental membrane in which the chorion has been removed. In another example, placental membrane or amnion tissue may refer to a placental membrane in which the epithelial layer has been removed.

As used herein, the terms “treatment” or “treating” include any desirable effect on the symptoms or pathology of a disease or condition, and may include even minimal reductions in one or more measurable markers of the disease or condition being treated. “Treatment” does not necessarily indicate complete eradication or cure of the disease or condition, or associated symptoms thereof. The subject receiving this treatment is any animal in need, including primates, in particular humans, and other mammals including, but not limited to, equines, cattle, swine, and sheep; and poultry and pets in general.

### B. Amniotic Tissue Preparation and Porous Collagen Matrices

1. Amniotic Tissue Preparation.

The amniotic tissue preparation includes amnion tissue and amniotic fluid cells. The amnion tissue component of the amniotic tissue preparation is produced from placentas collected from consenting donors in accordance with the Current Good Tissue Practice guidelines promulgated by the U.S. Food and Drug Administration. In particular, soon after the birth of a human infant via a Cesarean section delivery, the intact placenta is retrieved, and the placental membrane is dissected from the placenta. Afterwards, the placental membrane is cleaned of residual blood, placed in a bath of sterile solution, stored on ice and shipped for processing. Once received by the processor, the placental membrane is rinsed to remove any remaining blood clots, and if desired, rinsed further in an antibiotic rinse (Diaz-Prado S M, et al. Cell Tissue Bank 11, 183-195 (2010)).

The antibiotic rinse may include, but is not limited to, the antibiotics: amikacin, aminoglycosides, amoxicillin, ampicillin, ansamycins, arsphenamine, azithromycin, azlocillin, aztreonam, bacitracin, capreomycin, carbacephem, carbapenems, carbenicillin, cefaclor, cefadroxil, cefalexin, cefalotin, cefamandole, cefazolin, cefdinir, cefditoren, cefepime, cefixime, cefoperazone, cefotaxime, cefoxitin, cefpodoxime, cefprozil, ceftaroline fosamil, ceftazidime, ceftibuten, ceftizoxime, ceftobiprole, ceftriaxone, cefuroxime, chloramphenicol, ciprofloxacin, clarithromycin, clindamycin, clofazimine, cloxacillin, colistin, cycloserine, dapsone, daptomycin, demeclocycline, dicloxacillin, dirithromycin, doripenem, doxycycline, enoxacin, ertapenem, erythromycin, ethambutol, ethionamide, flucloxacillin, fosfomycin, furazolidone, fusidic acid, gatifloxacin, geldanamycin, gentamicin, glycopeptides, grepafloxacin, herbimycin, imipenem or cilastatin, isoniazid, kanamycin, levofloxacin, lincomycin, lincosamides, linezolid, lipopeptide, lomefloxacin, loracarbef, macrolides, mafenide, meropenem, methicillin, metronidazole, mezlocillin, minocycline, monobactams, moxifloxacin, mupirocin, nafcillin, nalidixic acid, neomycin, netilmicin, nitrofurans, nitrofurantoin, norfloxacin, ofloxacin, oxacillin, oxytetracycline, paromomycin, penicillin G, penicillin V, piperacillin, platensimycin, polymyxin B, pyrazinamide, quinolones, quinupristin/dalfopristin, rifabutin, rifampicin or rifampin, rifapentine, rifaximin, roxithromycin, silver sulfadiazine, sparfloxacin, spectinomycin, spiramycin, streptomycin, sulfacetamide, sulfadiazine, sulfamethizole, sulfamethoxazole, sulfanilamide, sulfasalazine, sulfisoxazole, sulfonamidochrysoidine, teicoplanin, telavancin, telithromycin, temafloxacin, temocillin, tetracycline, thiamphenicol, ticarcillin, tigecycline, tinidazole, tobramycin, trimethoprim, trimethoprim-sulfamethoxazole (co-trimoxazole) (TMP-SMX), and troleandomycin, trovafloxacin, or vancomycin.

The antibiotic rinse may also include, but is not limited to, the antimycotics: abafungin, albaconazole, amorolfin, amphotericin B, anidulafungin, bifonazole, butenafine, butoconazole, caspofungin, clotrimazole, econazole, fenticonazole, fluconazole, isavuconazole, isoconazole, itraconazole, ketoconazole, micafungin, miconazole, naftifine, nystatin, omoconazole, oxiconazole, posaconazole, ravuconazole, sertaconazole, sulconazole, terbinafine, terconazole, tioconazole, voriconazole, or other agents or compounds with one or more anti-fungal characteristics.

The placental membrane may be processed to remove one or more particular layers of the membrane. The chorion may be removed from the placental membrane by mechanical means well-known to those skilled in the art. The chorion may be removed, for example, by carefully peeling the chorion from the remainder of the placental membrane using blunt dissection (Jin C Z, et al. Tiss Eng 13, 693-702 (2007)). Removal of the epithelial layer from the placental membrane may be achieved using several methods well-known to those skilled in the art. The epithelial layer may be preserved or, if desired, may be removed by, for example, using trypsin to induce necrosis in the epithelial cells (Diaz-Prado S M, et al. Cell Tissue Bank 11, 183-195 (2010)). Removal of the epithelial layer may comprise, for example, treatment with 0.1% trypsin-ethylenediaminetetraacetic acid (EDTA) solution at 37° C. for 15 minutes followed by physical removal using a cell scraper (Jin C Z, et al. Tiss Eng 13, 693-702 (2007)). Preferably, the placental membrane utilized for the amnion tissue component of the amniotic tissue preparation is intact amniotic membrane including the epithelial cell layers, but with the chorion removed.

The placental membrane is morselized in an effort to preserve its structural properties using means known in the art including by cryogrinding. The average particle or particulate size of morselized placental membrane is about 250 micrometers, or in the range of 10 micrometers to 1000 micrometers. The morselized placental membrane includes organized amniotic extracellular matrix (ECM), amniotic tissue cells and growth factors contained within the ECM and amniotic tissue cells. The ECM includes amnion-derived collagen derived from the epithelium layer, the basement membrane layer, the compact layer, the fibroblast layer, the intermediate layer and the spongy layer of the amnion tissue. The ECM also includes fibronectin, laminin, proteoglycans and glycosaminoglycans.

The amniotic fluid cell component of the amniotic tissue preparation is prepared from amniotic fluid that is collected during amniocentesis or scheduled C-section from consenting donors. The amniotic fluid is spun thereby pelletizing the amniotic fluid cells. The resulting amniotic fluid cells are combined with the morselized placental membrane and stored in a solution containing approximately 5 to 10% vol/vol Dimethyl Sulfoxide (DMSO) and 15 to 25% vol/vol protein, with the balance being a crystalloid solution. The solution is then cryopreserved and stored within a vial at −140° C. until it is shipped from the manufacturer. It is then packed in dry ice and delivered to a hospital, all at a temperature of −80° C. A preferred amniotic tissue preparation is NuTech Medical's NuCel™ amniotic suspension.

2. Porous Collagen Matrix.

A collagen matrix is a three-dimensional scaffold comprising one or more forms of collagen including, but not limited to, for example, type I collagen, type II collagen, and type IV collagen. In addition, the collagen matrix may include one or more growth factors including, but not limited to, for example, TGF-β. A collagen matrix may be prepared using a variety of methodologies well-known to those skilled in the art. For example, a porous collagen matrix may be created by using pepsin-digested mammalian collagen that is neutralized with 1 M HEPES at pH 7.4, 1 M NaH—CO3, poured into a mold, frozen, lyophilized, and then irradiated [Zhou S, et al. Cell Tissue Bank 6, 33-44 (2005)]. The matrix may also be derived through treatments of an existing mammalian tissue, such as dermis or intestinal mucosa. The matrix may be prepared from collagen from bovine, ovine, porcine, or equine sources, although bovine collagen is commonly used.

When provided as a sheet, the collagen matrix can be derived, for example, from acellular, fetal bovine dermis, which is rich in Type III collagen. The collagen matrix sheet may also be composed of purified, cross-linked bovine Type I collagen and glycosaminoglycan (GAG). The matrix sheets may be a meshed, fenestrated or solid sheet composed of native, non-denatured collagen. When provided as a flowable matrix, the collagen matrix may composed of a granulated cross-linked bovine tendon collagen and GAG. The collagen matrix can be stored at room temperature for up to three years. Preferred collagen matrices are Integra Life Sciences Corporation's TenoGlide® collagen matrix sheet and Integra™ flowable matrix and dermal template sheet and TEI Biosciences' PriMatrix™ collagen matrix sheet. The collagen matrix can be produced in many different sizes, including large sizes suitable for large wounds, and shipped at ambient temperature. Thus the relatively much larger collagen matrix component of the preparation does not require shipment at cryopreservation, simplifying storage, shipment and associated logistics.

### B. Wound Care Treatment Preparation

1. Generally.

The amniotic tissue preparation is combined with the porous collagen matrix to form a wound care preparation including a portion of the amniotic tissue preparation adsorbed to the porous collagen matrix. To make the wound care preparation, the cryopreserved amniotic tissue preparation is emptied into a sterile basin to which an appropriate amount of saline is added. The frozen plug is allowed to thaw in the saline until all of the material is in a liquid state. Thereafter, as described in detail below, the porous collagen matrix is combined with the saline suspension of amniotic tissue thereby allowing the amniotic tissue to adsorb to the matrix.

Exemplary wound care treatments and the manufacture and analysis thereof are described below. In each instance, the wound care treatments include NuCel™ amniotic tissue adsorbed to a porous collagen matrix. NuCel™ amniotic tissue is an allograft product consisting of both amnion tissue and cells from the amniotic fluid. It includes microscopic particles of naturally occurring amnion tissue that contain organized amniotic extracellular matrix (ECM), cells, and growth factors contained within the ECM and cells. Specifically, the amnion contains a variety of ECM proteins including collagen, fibronectin, laminin, proteoglycans and glycosaminoglycans. The purpose of manufacturing and analyzing the following wound care treatments was to evaluate the appropriate conditions for adsorption of NuCel™ amniotic tissue into various porous collagen matrices including PriMatrix™ collagen matrix sheets, TenoGlide® collagen matrix sheets and Integra™ dermal template sheet and flowable matrix.

2. Adsorption of NuCel™ Amniotic Tissue to PriMatrix™ Collagen Matrix Sheet.

To evaluate the volume of fluid adsorbed to PriMatrix™ collagen matrix, 1×1 inch squares of a PriMatrix™ collagen matrix graft sheet were cut and then weighed. A graft sheet was saturated with 3 mL of phosphate buffered saline (PBS) for 5 minutes. Following adsorption, the residual liquid was allowed to drip away and the graft sheet was placed into a new plate and weighed. Using both dry and wet weights, it was calculated that PriMatrix™ collagen matrix adsorbed 800 μL of PBS per 1×1 inch square.

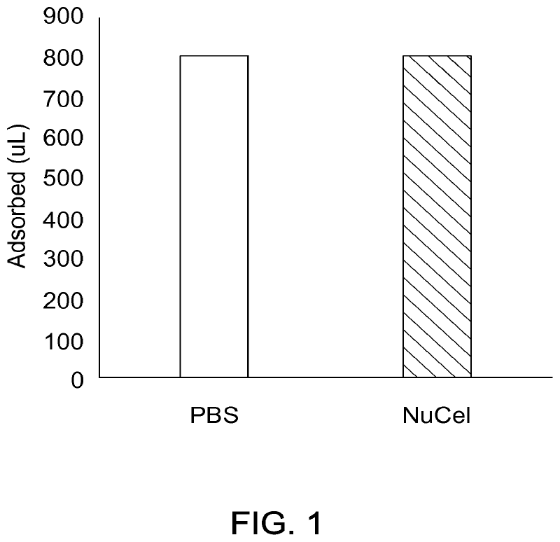

Adsorption of NuCel™ amniotic tissue on the PriMatrix™ collagen matrix graft material was measured. To do this, 1 mL of a saline suspension containing NuCel™ amniotic tissue was coated onto each 1×1 inch square of graft. Following coating for 5 minutes, the graft was moved to a new plate and wet weight measured. It was calculated that an average of 8024, of NuCel™ suspension adsorbed per 1×1 inch of graft. FIG. 1 is a graph comparing adsorption amounts of the saline suspension containing NuCel™ amniotic tissue and PBS solution to the 1×1 inch squares of PriMatrix™ collagen matrix sheets.

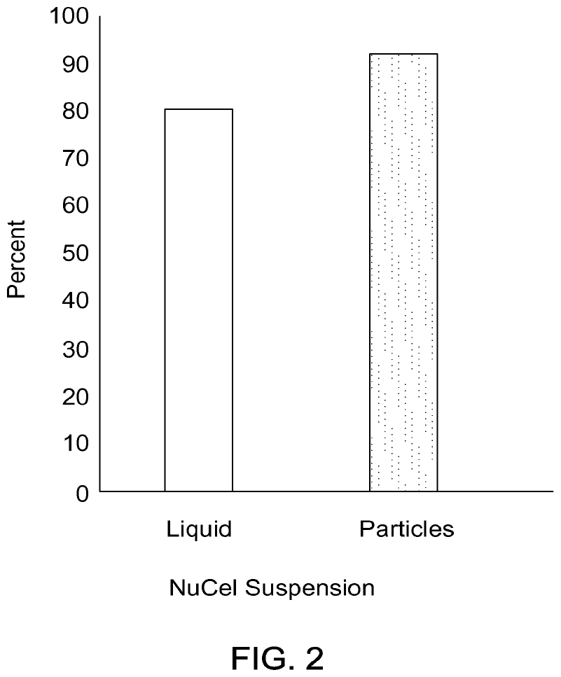

Adsorption of particulate, namely, amniotic fluid cells and morselized amniotic membrane, within the NuCel™ suspension was evaluated by evaluating the initial density of particulate in suspension and the density of particulate after coating the NuCel™ suspension onto the graft substrate. Following coating with 1 mL of the NuCel™ suspension, the graft was moved to a fresh plate and the plate used for coating was rinsed with PBS and evaluated for particulate density. It was found that when coating 1 mL of NuCel™ suspension onto a 1×1 inch square of PriMatrix™, approximately 80% of the liquid and 92% of the particles, namely, amniotic fluid cells and morselized amniotic membrane, originally present adsorbed to the graft. In sum, approximately 800 μL of the NuCel™ suspension can be stably adsorbed per 1×1 inch square of PriMatrix™. Additionally, the NuCel™ suspension adsorbed to PriMatrix™ in a relatively homogenous manner with liquid and particle phases resulting in 80% and 92% adsorption, respectively. FIG. 2 is a graph illustrating the respective percentages of saline and the particulate component (amniotic fluid cells and morselized placental membrane) of the saline solution containing NuCel™ amniotic tissue that adsorbed to the 1×1 inch square of PriMatrix™ collagen matrix sheet.

3. Adsorption of NuCel™ Amniotic Tissue to TenoGlide® Collagen Matrix Sheet.

To evaluate the volume of fluid adsorbed to TenoGlide® collagen matrix, a 2×2 inch TenoGlide® collagen matrix sheet was cut into four evenly sized 1×1 inch squares and placed into saline filled well plate to rinse away any leftover phosphate buffer. Saline was then removed from each well. In a separate well plate, a saline suspension of NuCel™ amniotic tissue was prepared that included 0.5 mL of NuCel™ amniotic tissue+0.5 mL of saline per well. The TenoGlide® collagen matrix sheet squares were picked up using forceps, and excess saline was allowed to drip away before the samples were moved to a new well plate containing the NuCel™ suspension. TenoGlide® collagen matrix sheet squares were soaked in NuCel™ suspension at room temperature for 20 minutes.

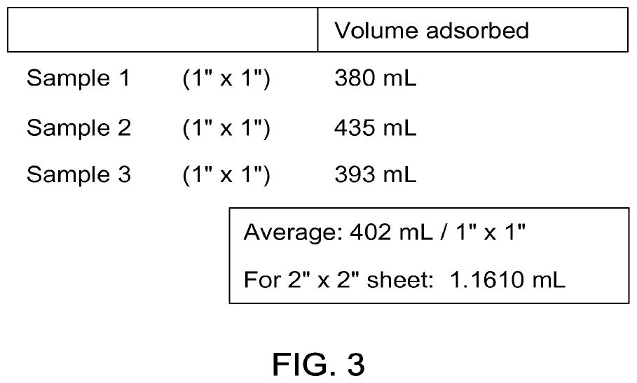

Following soaking, the TenoGlide® collagen matrix sheet squares were moved to a new well and excess NuCel™ suspension collected and measured to evaluate the volume of fluid adsorbed by each 1×1 inch square of TenoGlide® collagen matrix. Referring to FIG. 3, average adsorbed volume per 1×1 inch square was calculated to be 402 μL, corresponding to approximate necessary volume of at least 1.61 mL to hydrate the entire 2×2 inch square of TenoGlide® collagen matrix. When hydrating a 2×2 inch TenoGlide® collagen matrix sheet with NuCel™ suspension, the matrix is rinsed in sterile saline, allowing the saline to drip away from the matrix before applying NuCel™ suspension, preparing a NuCel™ suspension that consists of at least 850 μL of NuCel™ suspension mixed with an equal volume of saline, and hydrating TenoGlide® collagen matrix sheet with NuCel™ suspension for approximately 20 minutes. Adsorption of NuCel™ suspension onto pre-hydrated matrices is significantly enhanced by removing additional volume of adsorbed saline before coating with NuCel™ suspension by gently “wringing out” the matrix. Thus, a gentle wringing out step is preferred before coating TenoGlide® collagen matrix with NuCel™ suspension. This modestly increases the amount of NuCel™ amniotic tissue required.

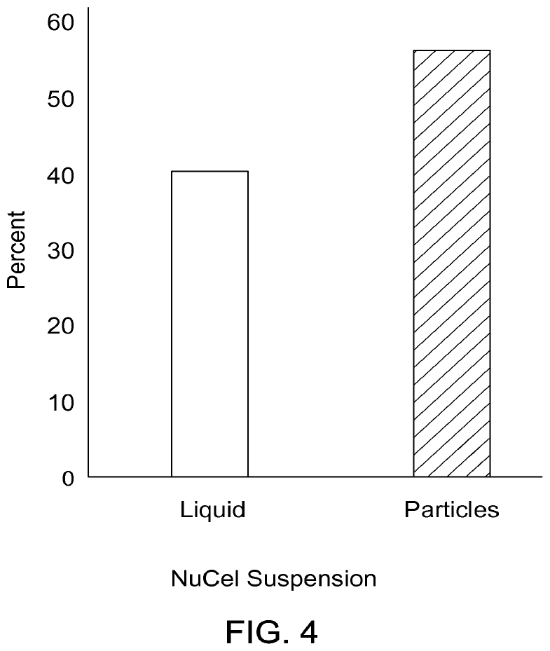

The adsorption of particulate from the NuCel™ suspension was evaluated. To do this, the initial density of particulate within the NuCel™ suspension was counted and recorded. The TenoGlide® collagen matrix sheet was soaked in the NuCel™ suspension for 20 minutes and TenoGlide® collagen matrix sheet removed from the plate. Using the portion of NuCel™ suspension that wasn't adsorbed, the volume of fluid and density of particulate post-adsorption was used to calculate the percentage of liquid and particulate (cells and amniotic membrane particles) adsorbed. Referring to FIG. 4, with an initial coating volume of 1 mL/1×1 inch square of TenoGlide® collagen matrix sheet, approximately 40% and 55% of liquid and particulate was adsorbed after 20 minutes, respectively.

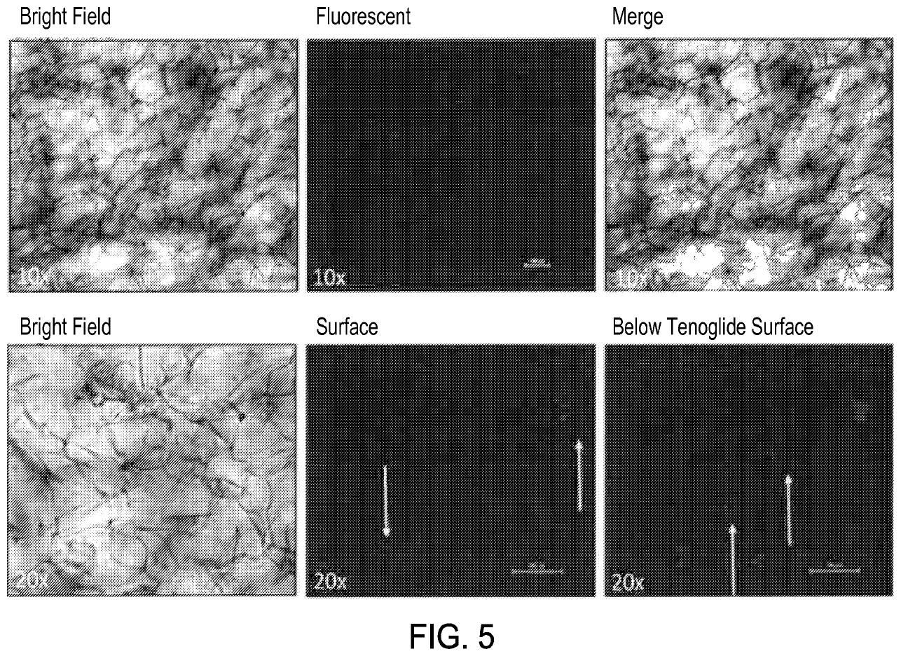

To demonstrate adsorption of NuCel™ amniotic tissue onto TenoGlide® collagen matrix, the NuCel™ amniotic tissue was labeled with 4′,6-diamidino-2-phenylindole (DAPI). NuCel™ amniotic tissue adsorption was then imaged using fluorescent microscopy. Referring to FIG. 5, the fluorescent and merged images demonstrated that amniotic fluid cells and amniotic membrane particles are co-localized with the TenoGlide® collagen matrix. Additionally, two separate focal planes within the same image were used to show that the amniotic fluid cells and particles were adsorbed not only on the surface but also further within the TenoGlide® collagen matrix.

4. Adsorption of NuCel™ Amniotic Tissue to Integra™ Flowable Collagen Matrix.

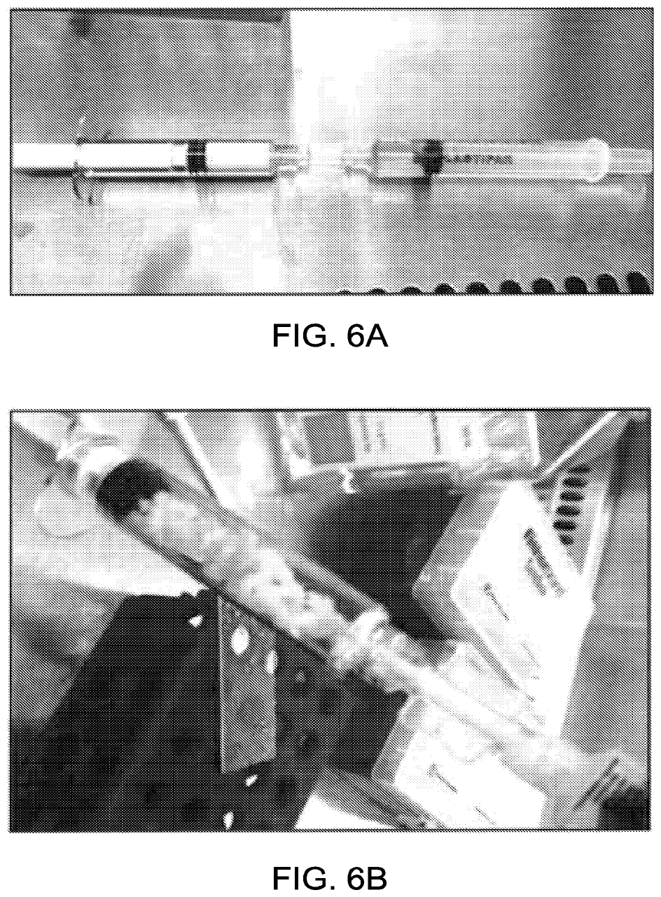

To evaluate the appropriate conditions for adsorption of NuCel™ amniotic tissue into Integra™ flowable collagen matrix, an attempt was made to remove the powdered Integra™ flowable collagen matrix from the syringe to calculate loading of NuCel™ amniotic tissue onto the flowable matrix. This was unsuccessful because the flowable particulate, when removed from the syringe, was lost to the air and difficult to weigh with accuracy. In a second attempt, as depicted in FIGS. 6A and 6B, a double syringe mechanism for mixing Integra™ flowable collagen matrix with a saline solution containing NuCel™ amniotic tissue was used with 1.5 mL of NuCel™ amniotic tissue and 1.5 mL of saline was used to hydrate the flowable matrix.

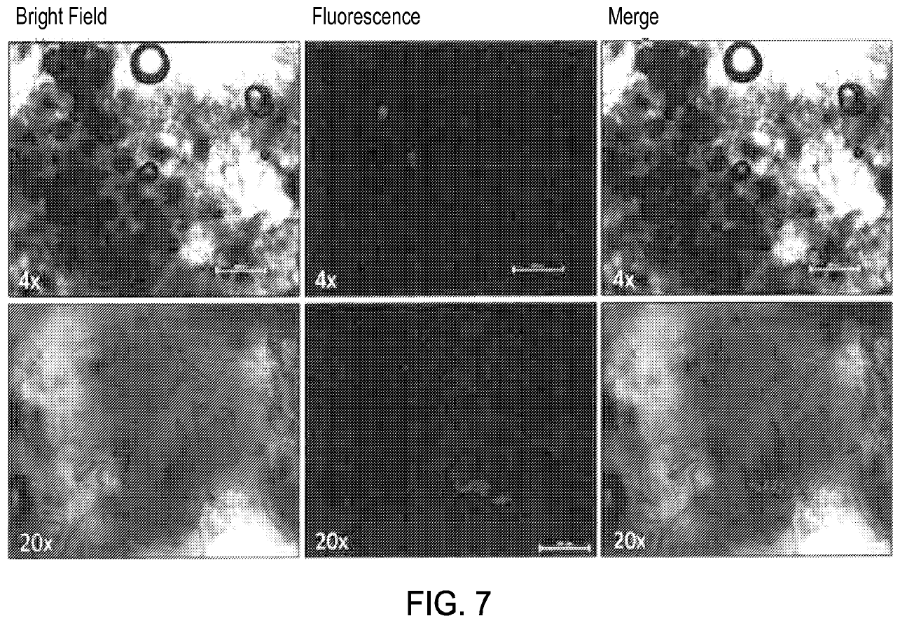

To confirm that the NuCel™ amniotic tissue was adsorbed within the flowable matrix, NuCel™ amniotic tissue cell nuclei and amniotic membrane particles were labeled with DAPI before mixing the Integra™ flowable collagen matrix with the NuCel™ amniotic tissue. Following labeling, the products were mixed using the double syringe mechanism and imaged using fluorescent microscopy. The fluorescent and merged images, as depicted in FIG. 7, clearly show NuCel™ amniotic tissue cell nuclei and amniotic membrane particles adsorbed onto the Integra™ flowable collagen matrix.

### C. Uses for the Wound Care Treatment Preparation

The embodiments of the preparations, described herein, may be used to promote healing of wounds and damaged or defective tissue.

1. Wound and Burn Care.

The treatment of large-scale wounds and burns and non-healing wounds is extremely challenging. Though many treatment options are available, some wounds are recalcitrant to treatment, and many do not heal with satisfactory results. Since at least 1910, amniotic membranes have been used in the treatment of burns and wounds. Accordingly, it is believed that the disclosed invention may be beneficially used in the treatment of non-healing wounds and burns, for example diabetic wounds or deep complex wounds involving muscle, tendon or bone. See, e.g., Jeng, J. C. et al., Seven Years' Experience With Integra as a Reconstructive Tool, (J Burn Care Res 2007; 28:120-126). An injectable embodiment may be packed or injected around or in the wound bed, filling deep wound areas which could not easily be filled by the thin, flat amniotic membranes in their normal orientation. An embodiment in matrix form may be used to cover large wound areas, which could not practicably be covered by placental membranes due to relatively small surface area available from each donor.

When treating wounds topically with the wound care treatment, the wound care treatment delivers from about 0.1 milligrams to about 3 milligrams of amniotic tissue per square centimeter of wound surface area.

2. Dermal Filler.

There are numerous cosmetic and plastic surgery applications in which dermal fillers may be used to restore the appearance of the skin. It is desirable that the products used be injectable, but sufficiently viscous to remain in place after injection. Numerous products have been investigated or used clinically. (Kontis, Theda C., Contemporary Review of Injectable Facial Fillers. Facial Plast Surg. 2013; 15(1): 58-64). Amniotic materials have been shown to support the repair and healing of the skin and connective tissue. The described embodiments, with or without the addition of other materials, such as, for example, hyaluronic acid, may be appropriately used as an injectable dermal filler.

1. A method of treating a wound comprising:
providing a first preparation including amnion tissue and amniotic fluid cells, wherein the amnion tissue is morselized amnion tissue and includes organized amniotic extracellular matrix (ECM), amniotic tissue cells and growth factors contained within the ECM and amniotic tissue cells,
providing a porous matrix including a mammalian collagen and optionally, glycosaminoglycan (GAG),
combining the first preparation with the porous matrix to form a second preparation, the second preparation including a portion of the first preparation adsorbed to the porous matrix, and
applying the second preparation to a mammal.

2. The method according to claim 1 wherein the first preparation is combined with the porous matrix to form the second preparation no more than about ten minutes prior to applying the second preparation to the wound.

3. The method according to claim 1 wherein the ECM includes amnion-derived collagen, fibronectin, laminin, proteoglycans and glycosaminoglycans.

4. The method according to claim 3 wherein the amnion-derived collagen is derived from an epithelium layer, a basement membrane layer, a compact layer, a fibroblast layer, an intermediate layer and a spongy layer of the amnion tissue.

5. The method according to claim 1 wherein the mammalian collagen is selected from the group consisting of Type I collagen, Type III collagen, a granulated cross-linked tendon collagen and combinations thereof.

6. The method according to claim 5 wherein the collagen is provided as a sheet, a meshed sheet or a flowable matrix.

7. The method according to claim 6 wherein, when the porous matrix is provided as the sheet, the first preparation adsorbs to the sheet in an amount of about 800 μl per square inch of the sheet.

8. The method according to claim 6 wherein, when the porous matrix is provided as the sheet including the GAG, the first preparation adsorbs to the sheet in an amount of about 300 μl to about 999 μl per square inch of the sheet.

9. The method according to claim 1 wherein the ground amnion tissue has an average particle size in a range of about 10 micrometers to about 1000 micrometers.

10. The method according to claim 1 further comprising removing a chorion layer from the amnion tissue.

11. The method according to claim 1 wherein the second preparation is applied to a skin wound of the mammal thereby promoting healing of the wound.

12. The method according to claim 11 comprising applying an effective amount of the second preparation to the wound, the effective amount including about 0.05 mg to about 5 mg of amnion tissue per square centimeter of the wound.

13. The method according to claim 1 wherein an effective amount of the second preparation is applied to a wound surface area, the wound surface area being at least ten times larger than a sum of the surface areas of an amnion side of each piece of the morselized amnion tissue.

14. A method of treating a wound comprising:
providing a first preparation including amnion tissue and amniotic fluid cells, wherein the amnion tissue is ground amnion tissue and includes amniotic extracellular matrix (ECM), amniotic tissue cells and growth factors and wherein the ECM includes fibronectin, laminin, proteoglycans, glycosaminoglycans and amnion-derived collagen that is derived from an epithelium layer, a basement membrane layer, a compact layer, a fibroblast layer, an intermediate layer and a spongy layer of the amnion tissue,
providing a porous matrix including a processed collagen, wherein the processed collagen is selected from the group consisting of Type I collagen, Type III collagen, a granulated cross-linked tendon collagen and combinations thereof,
combining the first preparation with the porous matrix to form a second preparation, the second preparation including a portion of the first preparation adsorbed to the porous matrix, and
applying the second preparation to the wound no later than 180 minutes after forming the second preparation, whereby the second preparation improves a healing rate of the wound.

15. The method according to claim 14 comprising providing the porous matrix as a sheet and adsorbing to the sheet between about 300 μl to about 999 μl of the first preparation per square inch of the sheet.

16. The method according to claim 14 comprising applying an effective amount of the second preparation to the wound, the effective amount including about 0.05 mg to about 5 mg of amnion tissue per square centimeter of the wound

17. The method according to claim 14 wherein the ground amnion tissue has an average particle size of about 250 micrometers.

18. A wound treatment comprising:
a porous collagen matrix, and
a preparation adsorbed to the porous collagen matrix, the preparation including amnion tissue and amniotic fluid cells, wherein the amnion tissue is ground amnion tissue and includes amniotic extracellular matrix (ECM), amniotic tissue cells and growth factors and wherein the ECM includes fibronectin, laminin, proteoglycans, glycosaminoglycans and amnion-derived collagen that is derived from an epithelium layer, a basement membrane layer, a compact layer, a fibroblast layer, an intermediate and a spongy layer of the amnion tissue.

19. The wound treatment according to claim 18 wherein, when the porous collagen matrix is in the form of a sheet, the preparation adsorbs to the sheet in an amount equal to about 300 μl to about 999 μl per square inch of the sheet.

20. The wound treatment according to claim 18 wherein the ground amnion tissue has an average particle size in a range of 10 micrometers to 1000 micrometers.

21. The wound treatment according to claim 18 wherein the amnion tissue excludes a chorion layer.

22. A method of treating a mammal comprising applying the wound treatment of claim 18 to a wound of a mammal thereby promoting healing of the wound.
